SEQUENCE LISTING (7 sequences)

SEQ ID NO: 1 - DNA, 2073 bp, Homo sapiens
  CDS (31)..(900)
cccacgcgtccggggcgtgctgcctgcaagatgtccgtgcgccgcggccggcggccggcgcggccggggacccgcctctcctggctgctgtgctgcagcgccctgctgtccccggccgcgggctacgtgatcgtgagctccgtgtcttgggccgtcaccaacgaggtggacgaggagctggacagcgcctccactgaggaggctatgcccgcgctgctagaggattcgggcagcatctggcagcaaagcttccccgcctctgcccacaaggaggacgcgcacctgcggccccgggcgggcgccgcccgggccaggccgccccccgcgccacccgggatgttctcctaccggcgcgagggcggccagacggccagtgcgcccccgggccctagactgcgcgccgccaccgcccgctccctggcccatgccagcgtctggggctgcctggccaccgtgtccacccacaagaagatccaaggactgccatttgggaactgcctgcccgtcagtgatggccccttcaacaatagcactgggattcctttcttctacatgacagccaaggaccccgtggtggctgatctgatgaagaaccccatggcctcgctgatgctgccagaatcagaaggggagttctgcagaaaaaacatcgttgatccggaagatccccgatgtgtccagttaacgctcactggccagatgatcgcagtgtctccagaagaagtagaatttgccaagcaagccatgttttcaaggcacccagggatgaggaagtggcctcgtcaatatgaatggttctttatgaagatgaggatagaacatatctggcttcagaaatggtatggaggcgcatccagtatttcaagggaggaatatttcaaagcagttcccagaaaggcctgatggagtgagaagaaagtccttggtgtttgcacttaaatgaaaaccttttcagtgatgcagccagacagctattgaccactgtctctttgttgaagggttcatagcagccctgccatccctgcagcagaatgagagagggtgaacagggaactctatgctagatttgagattaaagtggtcatttgcagatctccaactcacacagatacttcacgtagatagtctttattccattgtattcaatccagactcatcgattcagaaatcatataatagctggtggtcaaaatgacatgttgagatcattgttgtttcattgtttaaggaaaaaaaaaaatgcctgtacctaccaatggtgattgctttgtattgtgagagtatctttgttgcttgctctgccaaatgcagtcttggttctaagttcactgtgaccacgaagcagctgactgtgcatcacgcagtcacaatattgtttttagggtgagggtggaggactgtgtgtccgtggattactcctcctgctggtggattgcagatgcattattaggtcatactggctagaatgcagcttttctcccaccataacatgaaaacagtgtaagaacatagggtgctttgtgcatagcccttctctatgtaagcagccatggcagtcattaaagagaaaggagtagctttgacattaagctccccagatccctgctgctcatacttctggcaagggttcccctctctcatgcatgaacaggggcatccaaaataagaagctctccattctgtggtggggaaagcggagaggggagtgggtgaagctgggaaagtaaaggcagcacgttacagaaggaagaaaggaagccagtaactgagggcccactgcctgcccggccctgggccaggccctcaacagaagccatctcatttaagccctgcaaccaatgagatgcacgtcatcattggctcttacagacaagaaaactagactcagaggggctgagtccacatcccagacagctcactgcagacacaggtggagtggttcctacaagacatccagttttaacaacaaaagagttattgaaatgcatgggtagaaattgaaccaggaaaatcagtaaagtgattgtaaaaaagaaggactagcttgcctggagatgatgtttcttgcttttg

SEQ ID NO: 2 - protein, 290 aa, Homo sapiens
MSVRRGRRPARPGTRLSWLLCCSALLSPAAGYVIVSSVSWAVTNEVDEELDSASTEEAMPALLEDSGSIWQQSFPASAHKEDAHLRPRAGAARARPPPAPPGMFSYRREGGQTASAPPGPRLRAATARSLAHASVWGCLATVSTHKKIQGLPFGNCLPVSDGPFNNSTGIPFFYMTAKDPVVADLMKNPMASLMLPESEGEFCRKNIVDPEDPRCVQLTLTGQMIAVSPEEVEFAKQAMFSRHPGMRKWPRQYEWFFMKMRIEHIWLQKWYGGASSISREEYFKAVPRKA

SEQ ID NO: 3 - DNA, 867 bp, Mus musculus
  CDS (1)..(864)
atgtcgctgtccggcagggagcgtcctgcttggccagggagtcgcctgtcctggttgctatgctgcagcgccctgttgtccccagccgccggctacgtgatcgtgagctccgtgtcctgggctgtcaccaacgaggtagatgaggagctggacagtgcatccactgaggaggcgctgcccgcgctgctggaggactcgagtagcatctggcagcagagctttccggcctcggcgcacaaggaggacacgcacctgcgacctcggggctccgcccgcgccaggcccgcaccggccgcgcgtggcatgttctcctaccggcgggagagcggctcatctgaggcatcccctggccccagggtgcatgccggcaccgcccgctccctagcccacgctagctcctggggctgtctggccactgtgtccacccacgaaaagatccaaggactgccctttgggacctgcctggccatcagtgatggccccgtccacaacagcacagggatcccttttttttacatgacagccaaggaccctgcggtggctgacctggtgaagaatcccacagcctcgctggtgctgccggagtttgagggggagttttgcagaaaaaacatcgtcgacccagaagacccccgatgtgcccggttaacgctcaccggccggatggtcacggtgccaccaggggaggtggagttcgccaagcaagccatgttttcaaggcacccagggatgaggaagtggccccgacagtatgaatggttcttcatgaagatgtgggtagaacacatctggcttcacaaatggtatggaggtgtatctgacatcccgagggaggaatacttcaaggcagctccaaggaaggcctga

SEQ ID NO: 4 - protein, 288 aa, Mus musculus
MSLSGRERPAWPGSRLSWLLCCSALLSPAAGYVIVSSVSWAVTNEVDEELDSASTEEALPALLEDSSSIWQQSFPASAHKEDTHLRPRGSARARPAPAARGMFSYRRESGSSEASPGPRVHAGTARSLAHASSWGCLATVSTHEKIQGLPFGTCLAISDGPVHNSTGIPFFYMTAKDPAVADLVKNPTASLVLPEFEGEFCRKNIVDPEDPRCARLTLTGRMVTVPPGEVEFAKQAMFSRHPGMRKWPRQYEWFFMKMWVEHIWLHKWYGGVSDIPREEYFKAAPRKA

SEQ ID NO: 5 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 6 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 7 - protein, 210 aa, Homo sapiens
MGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

(19) United States
(12) Patent Application Publication
(10) Pub. No.: US 2007/0141024 A1
(43) Pub. Date: Jun. 21, 2007

(54) MAMMALIAN CYTOKINE; REAGENTS AND METHODS

(75) Inventors: Robert A. Kastelein, Redwood City, CA (US); Jochen Schmitz, Palo Alto, CA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/621,708

(22) Filed: Jan. 10, 2007

Related U.S. Application Data
(63) Continuation of application No. 10/857,457, filed on May 28, 2004, now abandoned.
(60) Provisional application No. 60/475,087, filed on May 30, 2003.

(51) Int. Cl. A61K 39/395; A61K 38/19; C07H 21/04; C12P 21/02; C07K 14/52; C07K 16/24

(52) U.S. Cl. 424/85.1; 435/69.5; 435/320.1; 435/325; 530/351; 530/388.23; 536/23.5; 424/145.1

(57) ABSTRACT

Provided are cytokines and methods of modulating activity of the immune system using cytokine agonists or antagonists. Also provided are methods of treatment and diagnosis of immune and proliferative disorders.

This application claims benefit of U.S. Provisional patent application Ser. No. 60/475,087 filed May 30, 2003.

### FIELD OF THE INVENTION

The present invention provides cytokine agonists and antagonists, and methods for treating and diagnosing immune, proliferative, and hematopoietic conditions. In particular, it provides purified proteins, binding compositions, nucleic acids, and related reagents and methods useful, e.g., in treating inflammatory and proliferative disorders.

### BACKGROUND OF THE INVENTION

The interleukin-1 (IL-1) family of cytokines contributes to the pathology of inflammatory disorders and proliferative conditions, e.g., arthritis and cancer. Cytokines of the IL-1 family include IL-1alpha (SEQ ID NO: 5), IL-1beta (SEQ ID NO: 6), IL-1delta, IL-1epsilon, basic fibroblast growth factor (SEQ ID NO: 7), IL-18, CREG and CREG2. IL-1alpha and IL-1beta are biosynthesized as 31 kDa polypeptides that are further processed to mature 17 kDa forms, while IL-1delta and L-1epsilon appear not to possess a distinct pro-form, see, e.g., Debets, et al. (2001) . J. Immunol. 167:1440-1446; McMahon, et al. (1997) J. Biol. Chem. 272:28202-28205; Irikura, et al. (2002) New Engl. J. Med. 169:393-398; Kim, et al. (2002) Biol. Chem. 277:10998-11003.

The IL-1 family also includes IL-1 receptors, i.e., IL-1RI, IL-1RII, and IL-1R accessory protein (a.k.a. IL-1R1, IL-1R2, and IL-1R3, respectively). IL-1alpha and IL-1beta trigger cell signaling by binding to IL-1R1, while IL-1RII can function as a molecule that absorbs circulating ligand (You, et al. (2001) New, Engl. J. Med. 193:101-109). IL-1 receptor antagonist (IL-1Ra), another IL-1 family protein. binds to IL-1 receptor without transmitting a signal and serves as an inhibitor of IL-1. IL-1ra and IL-1delta play similar roles in antagonizing signaling through receptors, i.e., IL-1ra antagonizes IL-1alpha-mediated signaling via IL-1R1, while IL-1delta antagonizes IL-1epsilon-mediated signaling via IL-1R6 Debets, et al. (2001) J. Immunol. 167:1440-1446; Apte and Voronov (2002) Sem. Cancer. Biol. 12:277-290; Wong, et al. (1997) Proc. Natl. Acad. Sci. USA 94:227-232).

IL-1 family members play a role in inflammatory conditions, e.g., rheumatoid arthritis, psoriasis, asthma, chronic obstructive pulmonary disorder (COPD), sepsis, and inflammatory bowel disorder (IBD) (Debets, et al. (1997) J. Immunol. 158:2955-2963; Chung (2001) Eur. Resp. J. Suppl. 34: 50s-59s; Freeman and Buchman (2001) Expert Opin. Biol. Ther. 1:301-308; Dinarello (2000) Chest 118:503-508). Rheumatoid arthritis (RA) is a common chronic inflammatory disorder characterized by degradation of joints, e.g., the synovial membrane, cartilage, and bone. The disorder strikes about 1% of the population and cannot be cured (Lacey, et al. (2003) Arthritis Rheum. 48: 103-109). IL-1 stimulates a number of cells involved in arthritic inflammation, e.g., fibroblasts, osteoclasts, chondrocytes, and neutrophils, which may show abnormal proliferation and release enzymes causing joint destruction, see, e.g., Krause, et al. (2002) J. Immunol. 169:6610-6616; Choy and Panayi (2001) New Engl. J. Med. 344:907-916; Woolley (2003) New Engl. J. Med. 348:1709-1711; Williams, et al. (2000) New Engl. J. Med. 164: 7240-7245; Feldmann and Maini (2001) Annu. Rev. Immunol. 19:163-196; Lacey, et al., supra; Niki, et al. (2001) J. Clin. Invest. 107:1127-1135; Attur, et al. (2000) J. Biol. Chem. 51:40307-40315).

Proliferative disorders are the second most common cause of death in the United States (Anderson (2002) National Vital Statistics Reports 50:1-86; Toribara and Sleisenger (2003) New Engl. J. Med. 332:861-867; Janne and Mayer (2000) New Engl. J. Med. 342:1960-1968; Fuchs and Mayer (1 995) New Engl. J. Med. 333:32-41). Cytokines of the L-1 family have been implicated in the control and pathology of proliferative disorders, i.e., cancer. IL-1 modulates progression through the cell cycle, e.g., by changing expression of cyclin-dependent kinases and cyclin-dependent kinase inhibitors. High doses of IL-1beta promote tumor invasiveness, while low doses can promote immune eradication of tumors, see, e.g., Zeisler, et al. (1998) Eur. J. Cancer 34:931-933; Yoshida, et al. (2002) Brit. J. Cancer 86-1396-1400; Nesbit, et al. (1999) Oncogene 18:6469-6476; Dinarello, et al. (1998)J. Leuko. Biol. 63:658-664; Apte and Voronov, supra; Saijo, et al. (2002) New Eng. J. Med. 169: 469-475; Murai, et al. (2001) J. Biol. Chem. 276:6797-6806; Koudssi, et al. (1998)J. Biol. Chem. 273: 25796-25803; Zeki, et al. (1999) J. Endocrinol. 160:67-73; Osawa, et al. (2000) J. Biochem. 127:883-893.

Although signaling pathways that control cell behavior in inflammation and proliferative disorders have been identified, these disorders remain poorly understood and new therapies are clearly needed. The present invention fulfills this need by identifying a cytokine associated with a number of inflammatory and proliferative conditions, including rheumatoid arthritis, ovarian cancer, and gastrointestinal cancer.

### BRIEF DESCRIPTION OF THE DRAWING

FIG. 1 shows aliment of IL-1 family members. IL-1alpha (SEQ ID) NO: 5) is from GenBank Accession No. ICHU1A. IL-1beta (SEQ ID) NO: 6) is from GenBank NP_{—}000567. Human basic fibroblast growth factor (SEQ ID NO: 7) is from GenBank NP_{—}001997. “Ident. or homol.” indicates where human IL-0beta shows identity or homology, as defined above, to at least one member of the IL-1 family cytokines shown.

### SUMMARY OF THE INVENTION

The present invention is based, in part, upon the recognition of the correlation of cytokine agonists with inflammatory and proliferative disorders.

The invention provides an isolated polypeptide comprising IL-0beta (SEQ ID NOs: 2 or 4), or an antigenic fragment thereof. Also provided is a fusion polypeptide or fusion peptide comprising the above polypeptide or fragment. Further provided is an isolated nucleic acid encoding a polypeptide comprising SEQ ID NOs: 2 or 4, or an antigenic fragment thereof, an expression or replicating vector comprising the above nucleic acid, and a host cell comprising the above vector.

Another embodiment of the invention provides a binding composition that specifically binds to a polypeptide comprising SEQ ID NOs: 2 or 4, or an antigenic fragment thereof the above binding composition derived from the antigen binding site of an antibody, where the above binding composition can comprise a human antibody; a humanized antibody; a monoclonal antibody; a polyclonal antibody; an Fab fragment or F(ab′)₂ fragment; or a detectable label.

Yet another aspect of the present invention provides a method of producing a polypeptide comprising SEQ ID NOs: 2 or 4, or an antigenic fragment thereof, comprising culturing the above-described host cell under conditions suitable for expression of the polypeptide, and isolating or purifying the polypeptide. Also encompassed is a method of modulating the activity of a cell comprising contacting the cell with an agonist of SEQ ID NOs: 2 or 4, or an antagonist of SEQ ID NOs: 2 or 4, as well as this method wherein the modulating is inhibiting, and this method wherein the agonist or antagonist comprises a binding composition derived from the antigen-binding site of an antibody that specifically binds to SEQ ID NOs: 2 or 4, or an antigenic fragment thereof.

In another embodiment, the invention contemplates a method of treating a subject suffering from a cellular disorder comprising treating with or administering an effective amount of an agonist of SEQ ID NOs: 2 or 4; or an antagonist of SEQ ID NOs: 2 or 4. Also provided is this method wherein the agonist or antagonist comprises a binding composition derived from the antigen-binding site of an antibody that specifically binds SEQ ID NOs: 2 or 4, or an antigenic fragment thereof, and this method wherein the binding composition comprises a human antibody; a humanized antibody; a monoclonal antibody; a polyclonal antibody; an Fab fragment or F(ab′)₂ fragment; or a detectable label. Further, the invention provides the above method wherein the cellular disorder is an immune or inflammatory disorder, or a proliferative condition, and the above method wherein the cellular disorder is rheumatoid arthritis or cancer, as well as the above method wherein the cancer is ovarian cancer, melanoma, gastrointestinal cancer, renal cancer, or prostate cancer.

Yet another aspect of the invention provides a method of diagnosing a cellular disorder comprising contacting a sample from a subject with a binding composition that specifically binds to a polypeptide comprising SEQ ID NOs: 2 or 4, or to an antigenic fragment thereof; or a nucleic acid that specifically hybridizes to a polynucleotide comprising SEQ ID NOs: 1 or 3.

The invention further contemplates a kit comprising a binding composition that specifically binds to a polypeptide comprising SEQ ID NOs: 2 or 4, or an antigenic fragment thereof; or a nucleic acid that specifically hybridizes to a nucleic acid comprising SEQ ID NOs: I or 3, and instructions for use.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

I. Definitions.

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

An “allelic variant” as used herein, is an alternative form of the gene encoding SEQ ID NOs: 1-4. Allelic variants may result from at least one mutation in the nucleic acid sequence and may result in altered mRNAs or in a polypeptide whose structure or function may or may not be altered. Any given alternative form of a gene may have none, one, or several allelic variants. Common mutational changes that give rise to allelic variants are generally ascribed to natural deletions, additions, or substitutions of nucleotides. Each of these types of changes may occur alone, or in combination with the others, one or more times in a given sequence.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, including selenomethionine, as well as those amino acids that are modified after incorporation into a polypeptide, e.g., hydroxyproline, gamma-carboxyglutamate, O-phosphoserine, and cystine.

“Antibody” refers to a polypeptide comprising a framework region from an immunoglobulin gene or fragments thereof that specifically recognizes and binds an antigen. The immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A “partially humanized” or “chimeric” antibody contains heavy and light chain variable regions of, e.g., murine origin, joined onto human heavy and light chain constant regions. A “humanized” or “fully humanized” antibody contains the amino acid sequences from the six complementarity-determining regions (CDRs) of the parent antibody, e.g., a mouse antibody, grafted to a human antibody framework. “Human” antibodies are antibodies containing amino acid sequences that are of 100% human origin, where the antibodies may be expressed, e.g., in a human, animal, bacterial, or viral host (Baca, et al. (1997) J. Biol. Chem. 272:10678-10684; Clark (2000) Immunol. Today 21:397-402).

Antibody fragments can be produced by digestion with various peptidases or by recombinant techniques. For example, pepsin digests an antibody below the disulfide linkages in the hinge region to produce F(ab′)₂, a dimer of Fab which itself is a light chain joined to V_{H}-C_{H}1 by a disulfide bond. The F(ab′)₂ can be reduced under mild conditions to break the disulfide linkage in the hinge region, thereby converting the F(ab′)₂ dimer into an Fab′ monomer. The Fab′ monomer is essentially Fab with part of the hinge region. “Fv” fragment comprises a dimer of one heavy chain and one light chain variable domain in tight association with each other. A single variable domain (or half of an Fv comprising only three CDRs specific for an antigen) has the ability to recognize and bind antigen, although at a lower affinity than the entire binding site. “Antibody” can refer to an antibody fragment produced by the modification of an intact antibody, to antibody compositions synthesized de novo using recombinant DNA methodologies, to single chain antibodies, to antibodies produced by phage display methods, and to monoclonal antibodies (U.S. Pat. No. 4,816,567 issued to Cabilly, et al.; U.S. Pat. No. 4,642,334 issued to Moore, et al.; Queen, et al. (I1989) Proc. Natl. Acad. Sci. USA 86:10029-10033; Kohler, et al. (1975) Nature 256:495-497).

“Monoclonal antibody” (mAb) refers to an antibody obtained from a population of substantially homogeneous antibodies, i.e., the individual antibody polypeptides comprising the population are identical except for possible naturally occurring mutations in the polypeptide chain that may be present in minor amounts, or to heterogeneity in glycosylation, disulfide formation, or folding. “Monoclonal antibody” does not suggest or limit any characteristic of the oligosaccharide component, or that there is homogeneity or heterogeneity with regard to oligosaccharide component. Monoclonal antibodies are highly specific, being directed against a single antigenic site or epitope. Polyclonal antibody preparations typically include different antibodies directed against different epitopes, while each mAb is directed against a single determinant on the antigen. In addition to their specificity, monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. “Monoclonal antibodies” also include clones of antigen-recognition and binding-site containing antibody fragments, such as those derived from phage antibody libraries.

“Diabody” refers to a fragment comprising a heavy chain variable domain (V_{H}) connected to a light chain variable domain (V_{L}) (Hollinger, el al. (1993) Proc. Natl. Acad. Sci. USA 90:6444-6448).

“Binding composition” refers to a molecule, small molecule, macromolecule, antibody, a fragment or analogue thereof or soluble receptor, capable of binding to a target. “Binding composition” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, which is capable of binding to a target. “Binding composition” may also refer to a molecule in combination with a stabilizer, excipient, salt, buffer, solvent, or additive, capable of binding to a target. “Binding” may be defined as an association of the binding composition with a target where the association results in reduction in the normal Brownian motion of the binding composition, in cases where the binding composition can be dissolved or suspended in solution.

“Cell line” refers to a population of cells capable of continuous or prolonged growth and division in vitro. Often, cell lines are clonal populations derived from a single progenitor cell. Spontaneous or induced changes can occur in the genome or can occur during storage or transfer of one or more cells present in the population of cells. Therefore, cells derived from the cell line referred to may not be precisely identical to the ancestral cells or cultures, and the cell line referred to includes such variants. The term “cell line” also includes immortalized cells (U.S. Pat. No. 6,090,611 issued to Covacci, et al.).

“Cellular disorder” refers, erg, to a disorder in development, maturation, growth, proliferation, adhesion, lifespan, location in the body, or concentration in a given physiological compartment, of a given type of cell, including immune cells. “Concentration” of a cell means, e.g., cells/unit volume of physiological fluid. “Cellular disorder” also refers to a disorder in response to stress, infection, or injury. Furthermore, cellular disorder may refer to a proliferative disorder, e.g., cancers, tumors, or pathological angiogenesis. Where a response of a normal cell, e.g., to stress, infection, injury, or cancer, produces pathological signs or symptoms in addition to that produced by the stress, infection, injury, or cancer alone, that response is a cellular disorder.

“Conservatively modified variants” applies to both amino acid and nucleic acid sequences. With respect to particular nucleic acid sequences, conservatively modified variants refers to those nucleic acids which encode identical or essentially identical amino acid sequences or, where the nucleic acid does not encode an amino acid sequence, to essentially identical nucleic acid sequences. Because of the degeneracy of the genetic code, a large number of functionally identical nucleic acids may encode any given protein.

As to amino acid sequences, one of skill will recognize that an individual substitution to a nucleic acid, peptide, polypeptide, or protein sequence which substitutes an amino acid or a small percentage of amino acids in the encoded sequence for a conserved amino acid is a “conservatively modified variant.” Conservative substitution tables providing functionally similar amino acids are well known in the art. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157: 105-132).

(1) Hydrophobic: Norleucine, Ile, Val, Leu, Phe, Cys, or Met;

(2) Neutral hydrophilic: Cys, Ser, Thr;

(3) Acidic: Asp, Glu;

(4) Basic: Asn, Gln, His, Lys, Arg;

(5) Residues that influence chain orientation: Gly, Pro;

(6) Aromatic: Trp, Tyr, Phe;

(7) Small amino acids; Gly, Ala, Ser.

“Exogenous” refers to substances that are produced outside an organism, cell, or human body, depending on the context. “Endogenous” refers to substances that are produced within a cell, organism, or human body, depending on the context.

An “expression vector” is a nucleic acid construct, generated recombinantly or synthetically, with one or more predetermined nucleic acid elements that permit transcription of a particular nucleic acid. Typically, the expression vector includes a nucleic acid to be transcribed operably linked to a promoter.

“Fusion protein or polypeptide” refers to a polypeptide chain synthesized from a nucleic acid, where the nucleic acid comprises an open reading frame encoding two or more polypeptide or peptide sequences, where the two or more nucleic acid sequences normally or generally do not naturally occur together to encode a single open reading frame. None, one, or all of the nucleic acids encoding the fusion protein may be of synthetic origin. The fusion protein may be synthesized by recombinant or synthetic methods, or it may occur naturally

“Truncated” protein or polypeptide refers to a polypeptide chain, e.g., synthesized from a nucleic acid, where the nucleic acid comprises an open reading frame encoding a polypeptide that lacks one or more consecutive amino acids at the N-terminus or C-terminus. “Lacks” in this context refers to comparison with a natural or synthetic non-truncated polypeptide. Truncated polypeptide also refers to a polypeptide that is modified, e.g., to introduce a start codon in the respective nucleic acid, or where the polypeptide is expressed from this start codon downstream of an existing start codon of the non-truncated polypeptide, where the truncation occurs at the N-terminus, or where the nucleic acid is modified to introduce a stop codon or stop site upstream from the start codon in the nucleic acid encoding the non-truncated polypeptide, where the truncation occurs at the C-terminus.

“Hybridization” that is specific or selective typically occurs when there is at least about 55% homology over a stretch of at least about 30 nucleotides, preferably at least about 75% over a stretch of about 25 nucleotides, and most preferably at least about 90% over about 20 nucleotides, see, e.g., Kanehisa (1984) Nucleic Acids Res. 12:203-213. Hybridization under stringent conditions, e.g., of a first nucleic acid to a second nucleic acid, are those that: (1) Employ low ionic strength and high temperature for washing, for example, 0.015 M sodium chloride/0.0015 M sodium citrate/0.1% sodium dodecyl sulfate at 50° C.; (2) Employ during hybridization a denaturing agent, such as formamide, for example, 50% (vol/vol) formamide with 0.1% bovine serum albumin/0.1% Ficoll® (Sigma-Aldrich, St. Louis, Mo.)/0.1% polyvinylpyrrolidone/50 mM sodium phosphate buffer at pH 6.5 with 750 sodium chloride, 75 mM sodium citrate at 42° C.; (3) Employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 ng/ml), 0.1% SDS, and 10% dextran sulfate at 42° C., with washes at 42° C. in 0.2×SSC and 0.1% SDS; or (4) Employ a buffer of 10% dextran sulfate, 2×SSC (sodium chloride/sodium citrate), and 50% formamide at 55° C., followed by a high-stringency wash consisting of 0.1×SSC containing EDTA at 55° C. (U.S. Pat. No. 6,387,657 issued to Botstein, et al.).

Stringent conditions for hybridization of nucleic acids are a function of salt, temperature, organic solvents, and chaotropie agents. Stringent temperature conditions will usually include temperatures in excess of about 30° C., more usually in excess of about 37° C., typically in excess of about 45° C., more typically in excess of about 50° C., preferably in excess of about 65° C., and more preferably in excess of about 70° C. Stringent salt conditions will ordinarily be less than about 1 M, more ordinarily less than about 500 mM, usually less than about 400 mM, more usually less than about 300 mM, typically less than about 200 mM, preferably less than about 100 mM, and more preferably less than about 80 mM, even down to less than about 20 mM. However, the combination of parameters is more important than the measure of any single parameter (Wetmur and Davidson (1968) J. Mol. Biol. 31:349-370).

An “immunoassay” is an assay that uses an antibody, or antigen-binding fragment thereof to specifically bind an antigen. The immunoassay is characterized by the use of specific binding properties of a particular antibody to isolate, target, detect, or quantify the antigen.

“Inhibitors” and “antagonists” or “activators” and “agonists” refer to inhibitory or activating molecules, respectively, e.g., for the activation of e.g., a ligand, receptor, cofactor, gene, cell, tissue, or organ. A “modulator” of gene activation, receptor signaling activity, cellular activity, and the like, is a molecule that is an inhibitor or an activator of the gene, receptor, or cell, respectively. The modulator may act alone, or it may use a cofactor, e.g., a protein, metal ion, or small molecule. Inhibitors are compounds that decrease, block, prevent, delay activation, inactivate, desensitize, or down regulate, e.g., a gene or protein. Activators are compounds that increase, activate, facilitate, enhance activation, sensitize, or up regulate, e.g., a gene, protein, ligand, or receptor. An inhibitor may also be defined as a composition that reduces, blocks, or inactivates a constitutive activity. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

To examine the extent of inhibition, samples or assays comprising a given gene or protein are treated with a potential activator or inhibitor and are compared to control samples without the inhibitor. Control samples, i.e., not treated with antagonist, are assigned a relative activity value of 100%. Inhibition is achieved when the activity value relative to the control is about 90%, typically 85%, more typically 80%, most typically 75%, generally 70%, more generally 65%, most generally 60%, often 55%, usually 50%, more usually 45%, most usually 40%, preferably 35%, more preferably 30%, still more preferably 25%, and most preferably less than 25%. Activation is achieved when the activity value relative to the control is about 110%, generally at least 120%, more generally at least 140%, most generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, of a cell, physiological fluid, tissue, organ, and animal or human subject, can be monitored by an endpoint. The endpoint may comprise a predetermined quantity or percentage of cell degranulation or secretion, e.g., of a cytokine, toxic oxygen, or a protease, or other indicator of inflammation. Alternatively, the endpoint may comprise a predetermined quantity of cellular ion flux, e.g., calcium flux; cell migration; cell adhesion; cell proliferation; potential for metastasis; cell differentiation; and change in phenotype, e.g., change in expression of gene relating to inflamation, apoptosis, transformation, cell cycle, or metastasis, see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4: 251-261; Robbins and Itzkowitz (2002) Med. Clin. North Am. 86:1467-1495; Grady and Markowitz (2002) Annul Rev. Genomics Hum. Genet. 3:101-128; Bauer, et al. (2001) Glia 36:235-243; Stanimirovic and Satoh (2000) Brain Pathol. 10:113-126). Generally, the endpoint of inhibition is at least 75% control, preferably the endpoint is at least 50% control, more preferably the endpoint is at least 25% control, and most preferably the endpoint is at least 10% control. Generally, the endpoint of activation is at least 150% control, preferably the endpoint is at least two times the control, more preferably the endpoint is at least four times the control, and most preferably the endpoint is at least 10 times the control.

“Detectable inhibition” or “detectable decrease,” e.g., in expression of a gene or polypeptide, or of a predetermined activity, refers, e.g., to a comparison of expression or activity in the presence and absence of an IL-0beta (SEQ ID NOs: 1-4) agonist, or in the presence or absence of an IL-0beta antagonist. “Detectable” may be a function of the context, e.g. of the reagents, instrumentation, or biological system.

A composition that is “labeled” is detectable, either directly or indirectly, by spectroscopic, photochemical, biochemical, immunochemical, isotopic, or chemical methods. For example, useful labels include 32P, ³³P, ³⁵S, ¹⁴C, ³H, ¹²⁵I, stable isotopes, fluorescent dyes, electron-dense reagents, substrates, or enzymes, e.g., as used in enzyme-linked immunoassays, or fluorettes (Rozinov and Nolan (1998) Chem. Biol. 5:713-728).

“Ligand” refers to an entity that specifically binds to a polypeptide or a complex of more than one polypeptide. A “ligand binding domain” is a region of a polypeptide that is able to bind to the entity. A ligand may be a peptide, soluble protein, membrane-associated protein, or integral membrane-bound protein. Where a ligand binds to a receptor, the question of which molecule is the ligand and which molecule is the receptor may be determined on a case-by-case basis. Generally, where the binding event results in cell signaling, the entity that is constitutively bound to the cell receiving the signal is considered to comprise the receptor, or comprise part of the receptor, and not the ligand. A freely diffusable and water-soluble entity that is involved in ligand/receptor interactions is usually a ligand, not a receptor.

“Peptide” refers to a short sequence of amino acids, where the amino acids are connected to each other by peptide bonds. A peptide may occur free or bound to another moiety, such as a macromolecule or a polypeptide. Where a peptide is incorporated into a polypeptide chain, the term “peptide” may still be used to refer specifically to the short sequence of amino acids. A “peptide” may be connected to another moiety by way of a peptide bond or some other type of linkage. A peptide is at least two amino acids in length and generally less than about 25 amino acids in length, where the maximal length is a function of custom or context. The terms “peptide” and “oligopeptide” may be used interchangeably.

“Protein” generally refers to the sequence of amino acids comprising a polypeptide chain. Protein may also refer to a three dimensional structure of the polypeptide. “Denatured protein” refers to a partially denatured polypeptide, having some residual three dimensional structure or, alternatively, to an essentially random three dimensional structure, i.e., totally denatured. The invention encompasses methods using polypeptide variants, e.g., involving glycosylation, phosphorylation, sulfation, disulfide bond formation, deamidation, isomerization, cleavage points in signal or leader sequence processing, covalent and non-covalently bound cofactors, oxidized variants, and the like. The formation of disulfide linked proteins are described, e.g., see Woycechowsky and Raines (2000) Curr. Opin. Chem. Biol. 4:533-539; Creighton, et al. (1995) Trends Biotechnol. 13:18-23.

The terms “percent identity” and “% identity” refer to the percentage of sequence similarity found by a comparison or alignment of two or more amino acid or nucleic acid sequences. Percent identity can be determined by a direct comparison of the sequence information between two molecules by aligning the sequences, counting the exact number of matches between the two aligned sequences, dividing by the length of the shorter sequence, and multiplying the result by 100. An algorithm for calculating percent identity is the Smith-Waterman homology search algorithm. See, e.g., Kann and Goldstein (2002) Proteins 48:367-376; Arslan, et al. (2001) Bioinformatics 17:327-337.

By “purified” and “isolated” is meant, when referring to a polypeptide, that the polypeptide is present in the substantial absence of the other biological macromolecules. The term “purified” as used herein means typically at least 70%, more typically at least 75%, most typically at least 80%, at least 85%, preferably at least 90%, more preferably at least 95%, most preferably at least 98% by weight, or greater, of biological macromolecules present. The weights of water, buffers, salts, detergents, reductants, protease inhibitors, stabilizers, and excipients, and molecules having a molecular weight of less than 1000, are generally not used in the determination of polypeptide purity (U.S. Pat. No. 6,090,611).

“Recombinant” when used with reference, e.g., to a nucleic acid, cell, virus, plasmid, vector, or the like, indicates modification by the introduction of an exogenous, non-native nucleic acid, alteration of a native nucleic acid, or by derivation in whole or in part from a recombinant nucleic acid, cell, virus, plasmid, or vector. Recombinant protein refers to a protein derived, e.g,, from a recombinant nucleic acid, virus, plasmid, vector, or the like.

“Soluble receptor” refers to receptors that are water-soluble and occur, e.g., in extracellular fluids, intracellular fluids, or weakly associated with a membrane. Soluble receptor also refers to receptors that have been released from tight association with a membrane, e.g., by limited proteolytic cleavage or cleavage of a lipid that maintains binding of the receptor to the membrane. Soluble receptor further refers to receptors that are engineered to be water soluble. The invention contemplates use of a soluble receptor to SEQ ID NOs: 2 or 4 for use in modulating the activity of IL-0beta, e.g., in the treatment of inflammation or cancer, see, e.g., Monahan, et al. (1997) New Engl. J. Med. 159:4024-4034; Moreland, et al. (1997) New Engl. J. Med. 337:141-147; Borish, et al. (1999) Am. J. Respir. Crit. Care Med. 160:1816-1823; Uchibayashi, et al. (1989) New Engl. J. Meg 142:3901-3908.

“Specifically” or “selectively” binds, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two fold greater, preferably at least ten times greater, more preferably at least 20-times greater, and most preferably at least 100-times greater than the affinity with any other antibody, or binding composition derived thereof. In a preferred embodiment the antibody will have an affinity that is greater than about 10⁹ liters/mol, as determined, e.g., by Scatchard analysis (Munsen, et al. (1980) Analyt. Biochem. 107:220-239).

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an IL-0beta agonist or IL-0beta antagonist to a human or animal subject, a cell, tissue, physiological compartment, or physiological fluid. “Treatment of a cell” also encompasses situations where the IL-0beta agonist or IL-0beta antagonist contacts IL-0beta or IL-0beta receptor, e.g., in the fluid phase or colloidal phase, but also situations where the agonist or antagonist does not contact the cell or the receptor.

II. General.

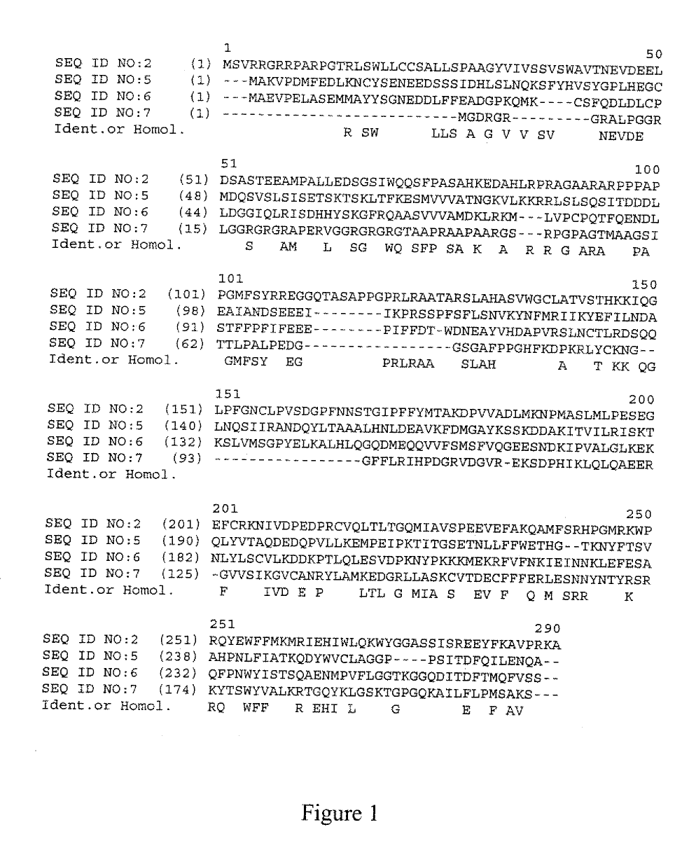

The present invention provides methods of using nucleic acids and polypeptide sequences of IL-0beta, a member of the IL-1 family. Homology between human IL-0beta (SEQ ID NO: 2), IL-1alpha (SEQ ID NO: 5), IL-1beta (SEQ ID NO: 6), and basic-FGF (SEQ ID NO: 7), is shown (FIG. 1).

The interleukin-1 (IL-1) family includes IL-1alpha (SEQ ID NO: 5), IL-1beta (SEQ ID NO: 6), IL-1Ra, IL-1delta, IL-1epsilon, basic fibroblast growth factor (a.k.a. basic-FGF; FGF-2) (SEQ ID NO: 7), and the IL-1 receptors: IL-1RI, IL-1RII, and IL-1R accessory protein (a.k.a. IL-1R1, IL-1R2, and IL-1R3, respectively). A family relationship between IL-1beta and basic-FGF was demonstrated by the identical three dimensional topology of the two proteins. The IL-1 family also includes IL-18. IL-1beta (SEQ ID NO: 6), IL-1Ra, IL-18, and basic fibroblast growth factor (SEQ ID NO: 7) have structures that are primarily beta-pleated sheet folded molecules, see, e.g., Zhang, et a. (1991) Proc. Natl. Acad. Sci. USA 88:3446-3450; Powers, et al. (2000) Endocrine-Related Cancer 7:165-197; Dinarello, et al. (1998) J. Leuko. Biol. 63:658-664; Zhang, et al. (1991) Proc. Natl. Acad. Sci. USA 88:3446-3450; Dinarello (1996) Blood 87:2095-2147; Dinarello (1997) Cytokine Growth Factor Revs. 58: 253-265; Dinarello (1998) Intern. Rev. Immunol. 16:457-499.

Motifs residing in human SEQ ID NO. 2 were determined using the Prosite database (Falquet, et al. (2002) Nucl. Acids Res. 30:235-238; Nicodeme, et al. (2002) Bioinformatics 18:S161-S171). These motifs include, e.g., an N-terminal amidation site at amino acids 5-8 of SEQ ID NO: 2, N-glycosylation sites at residues 165 and 166 of SEQ ID NO: 2, sites for protein kinase C phosphorylation at amino acids 2, 105, 126, 144, and 176 of SEQ ID NO: 2, and casein kinase II phosphorylation sites at residues 54, 176, 198, 228, and 278 of SEQ ID NO: 2. N-glycosylation sites at Asn-165 and Asn-166 of SEQ ID NO: 2 have been identified by Kunita, et al. (2002) Genomics 80:456-460). Within IL-0beta occur a number of Arg-Pro and Gly-Pro sites, which are potential substrates of postproline aminopeptidases, enzymes that process chemokines and growth factors (Abbott, et al. (2000) Eur. J. Biochem. 267:6140-6150).

Members of the IL-1 family can accumulate and function in multiple locations, e.g., in the nucleus and extracellular fluids. SEQ ID NO: 2 (a.k.a. IL-0beta; human CREG2; GenBank NP_{—}722578; BAC22189) shows homology to human IL-0alpha (a.k.a. CREG; GenBank AAC34861; NP_{—}003842), a protein that appears to be secreted as well as to locate in the nucleus (Kunita, et al., supra, Veal, et al. (1998) Mol. Cell. Biol. 18:5032-5041; Veal, et al. (2000) Oncogene 19:2120-2128). IL-0beta also is found to be secreted and to be located in an intracellular organelle (Kunita, et al., supra).

IL-1beta functions extracellularly, while evidence suggests it also has an intracellular function. Basic fibroblast growth factor (SEQ ID NO: 7) accumulates in the cytosol, nucleus, cell surface, and extracellular space Perregaux, et al. (2002) J. Immunol. 168:3024-3032; Tatsuta, et al. (1996) J. Immunol. 157:3949-3957; Estival, et al. (1996) J. Biol. Chem. 271:5663-5670; Arese, et al. (1999) Mol. Biol. Cell 10:1429-1444; Trudel, et al. (2000) J. Cell. Physiol. 185:260-268).

III. Immune and Proliferative Disorders.

The invention finds use in the modulation and diagnosis of immune and proliferative disorders. IL-1 family members have been implicated in inflammatory conditions, e.g., rheumatoid arthritis, asthma, chronic obstructive pulmonary disorder (COPE)), sepsis, inflammatory bowel disorder (IBD), and Alzheimer's disease (Debets, et al. (1997) J. Immunol. 158:2955-2963; Chung (2001) Eur. Resp. J. Suppl. 34:50s-59s; Freeman and Buchman (2001) Expert Opin. Biol. Ther. 1:301-308; Dinarello (2000) Chest 118:503-508). The IL-1 cytokines are expressed by and mediate the activity of, e.g., lymphocytes, macrophages, monocytes, synovial cells, fibroblasts, and endothelial cells. These cells can respond to IL-1 by proliferation or by producing cytokines and other mediators of inflammation. For example, IL-1 is high in arthritic joints, while treatment with IL-1 antagonists reduces the severity of arthritis, as shown by studies of humans and animal models (Joosten, et al. (2003) Arthritis Rheumatism 48:339-347; Kawashima and Miossec (2003) Arthritis Rheumatism 48:631-637; Iwakura (2002) Cytokine Growth Factor Revs. 13:341-355; Feldman, et al. (1996) Annu. Rev. Immunol. 14:397-400).

IL-1 family members are involved in proliferative disorders, e.g., ovarian cancer, melanoma, and renal carcinoma (Nesbit, et al. (1999) Oncogene 18:6469-6476; Zeisler, et al. (1998) Eur. J. Cancer 34:931-933; Triozzi, et al. (1995) J. Clin. Oncol. 13:482-489; Yoshida, et al. (2002) Brit. J. Cancer 86:1396-1400; Zeisler, et al. (1998) Eur J. Cancer 34:931-933). Common ovarian cancers include malignant serous tumors, such as, adenocarcinoma, papillary adenocarcinoma, and papillary cystadenocarcinoma, malignant mucinous tumors, and malignant endometrioid tumors (Hefler, et al. (2002) J. Soc. Gynecol. Investig. 9:386-390; DeVita, et al. (eds.) (2001) Cancer Principles and Practice of Oncology, 6^{th} ed., Lippincott, Williams, and Wilkins, Phila., pp. 1597-1625). Increased cytokines in cancer tissue may be expressed by cancer cells and/or infiltrating immune cells, see, e.g., Kowalczyk, et al. (1997) Br. J. Urol. 80:543-547; Demaria, et al. (2001) Clin. Cancer Res. 7:3025-3030).

Proliferative disorders result from alterations in the control, e.g., of the cell cycle and apoptosis. Various stress factors such as γ-irradiation and serum starvation/refeeding influence these regulated responses. γ-Irradiation can result in growth arrest or apoptosis, depending on the cell type or on the environment (Komarova and Gudkov (1998) Sem. Cancer Biol. 8:389-400). IL-1 is modulated by γ-irradiation where expression can be increased or decreased, depending on the conditions (Zhou, et al. (2001) Int. J. Radiat. Biol. 77:763-772; Ibuki and Goto (1999) J. Radiat. Res. 40:253-262; Bruserud and Ulvestad (1999) J. Hematother. Stem Cell Res. 8:431-441).

The invention contemplates use of SEQ ID NOs: 1-4 for the treatment and diagnosis of inflammatory disorders, e.g., rheumatoid arthritis or Alzheimer's disease.

Also provided is use of SEQ ID NOs: 1-4 for the treatment and diagnosis of cell proliferation and proliferative conditions e.g., melanoma, or ovarian, renal,. or colon cancer.

IV. Analogs of IL-0beta.

This invention provides nucleic acids, proteins, and peptides having substantial sequence identity to SEQ ID NOs: 1-4, antigenic fragments thereof, and binding composition thereto, including polymorphic variants, allelic variants, and variants due to mutations and alternative splicing.

SEQ ID NOs: 1-4 modified by recombinant or chemical methods is provided. Mutagenesis can be conducted by making amino acid substitutions, insertions, or deletions. Substitutions, deletions, insertions, or any combinations thereof maybe generated to arrive at a final construct. Insertions include amino- or carboxy-terminal fusions.

Fusion polypeptides comprising IL-0beta, or a fragment thereof, as well as the nucleic acids encoding them, can be made by a number of methods (Sambrook, et al. (1989) Molecular Cloning: A Laboratory Manual (2d ed.), vols. 1-3, Cold Spring Harbor Laboratory; and Ausubel, et al. (eds.) (1993) Current Protocols in Molecular Biology, Greene and Wiley, NY; Godowski, et al. (1988) Science 241:812-816; Rais-Beghdadi, et al. (1998) Appl. Biochem. Biotechnol. 74:95-103; U.S. Pat. No. 4,859,609).

The invention contemplates SEQ ID NOs: 2 or 4 polypeptides modified in oligosaccharide or lipid identity, content, or location, see, e.g., Elbein (1987) Ann. Rev. Biochem. 56:497-534; Summers (1988) Bio/Technology 6:47-55; and Kaufman (1990) Meth. Enzymol. 185:487-511; Low (1989) Biochim. Biophys. Acta 988:427-454; Tse, et al. (1985) Science 230:1003-1008; Brunner, et al. (1991)J. Cell Biol. 114:1275-1283). The fusion protein of the present invention may comprise peptides that facilitate purification, e.g., streptavidin-binding peptides, His tags, and FG fragments; peptides that provide an epitope, e.g., a FLAG tag; peptides that facilitate binding to other peptides or polypeptides, e.g., leucine zippers; and peptides that target the invention to a specific receptor or cell, e.g., fusion proteins comprising an antibody. The fusion protein may also comprise linked antigen-binding regions derived from an antibody, e.g., a bifunctional antibody or a chain of Fv fragments (Joosten, et al. (2003) Microbial Cell Factories 2:1-15; Helguera, et al. (2002) Clin. Immunol. 105:233-246; Liu, et al. (2001) Curr. Protein Peptide Sci. 2:107-121).

The invention further contemplates immobilization, e.g., to a bead, magnetic bead, slide, microarray, fabric, polymer, or device such as a lab on a chip. See, e.g., U.S. Pat. Nos. 6,176,962 and 6,517,234. The invention contemplates immobilized nucleic acids, polypeptides, peptides, antibodies and antibody fragments, as well as other reagents.

V. Screening for IL-0beta Expression and for Therapeutic Agents.

Cells or animals can be screened for SEQ ID NOs: 1-4 expression. Expression of mRNA is measured by, e.g., hybridization or the polymerase chain reaction (PCR) (Liu, et al. (2002) Analyt. Biochem. 300:40-45; Huang, et al. (2000) Cancer Res. 60:6868-6874; Wittwer, et al. (1997) Biotechniques 22:130-138; Schmittgen, et al. (2000) Analyt. Biochem. 285:194-204; Heid, et al. (1996) Genome Res. 6:989-994; Sims, et al. (2000) Analyt. Biochem. 281:230-232).

Cells and microarrays of nucleic acids can be used for screening (Ausubel, et al. (2001) Curr. Protocols Mol. Biol., Vol. 4, John Wiley and Sons, New York, N.Y., pp. 22.0.1-22.3.26; Huang, et al. (2000) Cancer Res. 60:6868-6874; Ausubel, et al. (2001) Curr. Protocols Mol. Biol. Vol. 4, John Wiley and Sons, New York, N.Y., pp. 25.0.1-25B.2.20 and Ausubel, et al. (2001) Curr. Protocols Mol. Biol., Vol. 3, John Wiley and Sons, New York, N.Y., pp. 140.1-14.14.8).

The above techniques can also be used for screening of therapeutic agents that modulate the expression, processing, secretion, and binding functions of SEQ ID NOs: 1-4.

VI. Purification of Proteins and Nucleic Acids.

It is contemplated to purify the polypeptide and nucleic acid diagnostics or therapeutics of the invention by methods that are established in the art. Purification can involve ion exchange chromatography, immunoprecipitation, epitope tags, affinity chromatography, high pressure liquid chromatography, and use of stabilizing agents, detergents or emulsifiers (Dennison and Lovrien (1997) Protein Expression Purif. 11:149-161; Murby, et al. (1996) Protein Expression Purif. 7:129-136; Ausubel, et al. (2001) Curr. Protocols Mol. Biol., Vol. 3, John Wiley and Sons, New York, N.Y., pp. 17.0.1-17.23.8; Rajan, et al. (1998) Protein Expression Purif. 13:67-72; Amersham-Pharmacia (2001) Catalogue, Amersham-Pharmacia Biotech, Inc., pp. 543-567, 605-654; Gooding and Regnier (2002) HPLC of Biological Molecules, 2^{nd} ed., Marcel Dekker, NY).

VII. Agonists; Antagonists; Antibodies.

Antibodies and binding compositions derived from an antigen-binding site of an antibody are provided. These include human and humanized antibodies, monoclonal and polyclonal antibodies, and binding fragments, such as Fab, F(ab)₂, and Fv fragments, and engineered versions thereof. “Derived” includes derived by chemical modification of an antibody, as well as derived by de novo synthesis of a binding composition with modeling of the binding composition after the structural features of the antibody. The antibody or binding composition can be agonistic or antagonistic. Antibodies that simultaneously bind to a ligand and receptor are contemplated. Monoclonal antibodies will usually bind with at least a K_{D} of about 1 mM, more usually at least about 300 μM, typically at least about 100 μM, more typically at least about 30 μM, preferably at least about 10 μM, and more preferably at least about 3 μM or better.

Antigenic regions of human IL-0beta (SEQ ID NO: 2) can be used for preparing antibodies. Regions of increased antigenicity include, e.g., amino acids 64-78 and 96-111 of SEQ ID NO: 2. The invention provides a nucleic acid encoding a polypeptide comprising amino acids 64-78 and/or 96-111 of SEQ ID NO: 2, a polypeptide comprising amino acids 64-78 and/or 96-111 of SEQ ID NO: 2, and a binding composition that specifically binds a polypeptide comprising amino acids 64-78 and/or 96-111, of SEQ ID NO: 2. Also provided is a binding composition that specifically binds to a polypeptide comprising amino acids 1-134 of SEQ ID NO: 2.

Regions of murine IL-0beta (SEQ ID NO: 4) of increased antigenicity include, e.g., amino acids 60-88 and 96-110 of SEQ ID NO: 4 (Vector NTI® Suite, Informax, Inc., Bethesda, Md.). The invention provides a nucleic acid encoding a polypeptide comprising amino acids 60-68 and/or 96-110 of SEQ ID NO: 4, a polypeptide comprising amino acids 60-68 and/or 96-110 of SEQ ID NO: 4, and a binding composition that specifically binds a polypeptide comprising amino acids 60-68 and/or 96-110 of SEQ ID NO: 4.

Monoclonal, polyclonal, and humanized antibodies can be prepared, see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205-6213; He, et al. (1998) J. Immunol. 160:1029-1035; Tang, et al. (1999) J. Biol. Chem. 274:27371-27378). A humanized antibody contains the amino acid sequences from six complementarity determining regions (CDRs) of the parent mouse antibody, which are grafted on a human antibody framework.

Alternatives to humanization include use of fully human antibodies, as well as human antibody libraries displayed on phage or human antibody libraries contained in transgenic mice (Vaughan, et al. (1996) Nat. Biotechnol. 14:309-314; Barbas (1995) Nature Med. 1:837-839; de Haard, et al. (1999) J. Biol. Chem. 274:18218-18230; McCafferty et al. (1990) Nature 348:552-554; Clackson et al. (1991) Nature 352:624-628; Marks et al. (1991) J. Mol. Biol. 222:581-597; Mendez, et al. (1997) Nature Genet. 15:146-156; Hoogenboom and Chames (2000) Immunol. Today 21:371-377; Barbas, et al. (2001) Phage Display: A Laboratory Manual, Cold Spring Harbor Laboratory Press, Cold Spring Harbor, N.Y.; Kay, et al. (1996) Phage Display of Peptides and Proteins: A Laboratory Manual, Academic Press, San Diego, Calif.; de Bruin, et al. (1999) Nat. Biotechnol. 17:397-399).

Single chain antibodies, single domain antibodies, and bispecific antibodies are described (Malecki, et al. (2002) Proc. Natl. Acad. Sci. USA 99:213-218; Conrath, et al. (2001) J. Biol. Chem. 276:7346-7350; Desmyter, et al. (2001) J. Biol. Chem. 276:26285-26290, Kostelney, et al. (1992) New Engl. J. Med. 148:1547-1553; U.S. Pat. Nos. 5,932,448; 5,532,210; 6,129,914; 6,133,426; 4,946,778).

Antigen fragments can be joined to other materials, such as fused or covalently joined polypeptides, to be used as immunogens. An antigen and its fragments may be fused or covalently linked to a variety of immunogens, such as keyhole limpet hemocyanin, bovine serum albumin, or ovalbumin (Coligan, et al. (1994) Current Protocols in Immunol., Vol. 2, 9.3-9.4, John Wiley and Sons, New York, N.Y.). Peptides of suitable antigenicity can be selected from the polypeptide target, using an algorithm, such as those of Parker, et al. (1986) Biochemistry 25:5425-5432; Jameson and Wolf (1988) Cabios 4:181-186; or Hopp and Woods (1983) Mol. Immunol. 20:483-489.

Purification of antigen is not necessary for the generation of antibodies. immunization can be performed by DNA vector immunization. See, e.g., Wang, et al. (1997) Virology 228: 278-284. Alternatively, animals can be immunized with cells bearing the antigen of interest. Splenocytes can then be isolated from the immunized animals, and the splenocytes can fused with a myeloma cell line to produce a hybridoma. Resultant hybridomas can be screened for production of the desired antibody by functional assays or biological assays, that is, assays not dependent on possession of the purified antigen. Immunization with cells can prove superior for antibody generation than immunization with purified antigen (Maynard, et al. (1997) Immunity 7:283-290; Wright, et al. (2000) Immunity 13:233-242; Preston, et al. (1997) Eur. J. Immunol. 27:1911-1918; Kaithamana, et al. (1999) New Engl. J. Med. 163:5157-5164).

Antibody to antigen binding properties can be measured, e.g., by surface plasmon resonance or enzyme linked immunosorbent assay (ELISA) (Neri, et al. (1997) Nat. Biotechnol. 15:1271-1275; Jonsson, et al. (1991) Biotechniques 11:620-627; Hubble (1997) Immunol. Today 18:305-306). The antibodies of this invention can be used for affinity chromatography in isolating the antibody's target antigen and associated bound proteins. See, e.g., Wilchek, et al. (1984) Meth. Enzymol. 104:3-55.

Antibodies to variants of SEQ ID NOs: 2 or 4 possessing substitutions that do not substantially affect the functional aspects of the nucleic acid or amino acid sequence, are within the definition of the contemplated invention. Variants with truncations, deletions, additions, and substitutions of regions which do not substantially change the biological functions of these nucleic acids and polypeptides are also within the definition of the contemplated invention.

VIII. Receptors.

The invention contemplates modulating interactions between SEQ ID NOs: 2 or 4 and an IL-0beta receptor (IL-0betaR). IL-0betaR is identified by binding of SEQ ID NOs: 2 or 4 to a cell. IL-0beta R is purified by binding IL-0beta to a cell, releasing the IL-0beta/IL-0betaR complex from the cell membrane, e.g., with detergent, and immunuoprecipitation of the IL-0beta/IL-0betaR complex, or by using labeled IL-0beta as a reagent for monitoring chromatographic migration and purity of solubilized IL-0betaR. IL-0betaR is also identified by immunization with cells or cell extracts bearing IL-0betaR, preparation of hybridoma clones, and screening the hybridomas for antibodies that serve as agonists or antagonists of IL-0beta.

IX. Therapeutics and Pharmaceutical Compositions.

The invention provides methods to treat and diagnose various inflammatory disorders, e.g., rheumatoid arthritis or Alzheimer's disease, and proliferative disorders, e.g., ovarian or colon cancer. Formulations of antibodies, binding composition, polypeptides, antibody mimetics, or small molecule therapeutics are prepared for storage by mixing with physiologically acceptable carriers, excipients, or stabilizers in the form of, e.g., lyophilized powders, slurries, aqueous solutions, see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.; U.S. Pat. Nos. 6,096,728; 6,342,220; and 5,440,021. Therapeutic reagents and methods based on nucleic acids are provided, e.g., anti-sense treatment using an anti-sense nucleic acid based on SEQ ID NOs: 1 or 3 (Herweijer and Wolff (2003) Gene Ther. 10:453-458; Gleave, et al. (2002) Cancer Metastasis Rev. 21:79-92; Devi (2002) Curr. Opin. Mol. Ther. 4:138-148; Gleave, et al. (2002) Curr. Drug Targets 4:209-221).

Therapeutic compositions comprising an agonist, antagonist, binding composition, or small molecule, can be administered, e.g., by systemic, intraperitoneal, intramuscular, dermal, subcutaneous, oral, nasal, pulmonary, suppository, and intratumor routes. Sustained-release preparations, liposomes, aerosols, or viral vectors may supply the therapeutic composition by the contemplated method (Sidman et al. (1983) Biopolymers, 22:547-556; Langer et al. (1981) J. Biomed. Mater. Res. 15:167-277; Langer (1982) Chem. Tech. 12:98-105; Lasic and Papahadjopoulos (eds.) (1998) Medical Applications of Liposomes, Elsevier Health Sciences, Phila., Pa.; Janoff (ed.) (1999) Liposomes: Rational Design, Marcel Dekker, Inc., NY, N.Y.; Knowles, et al. (1995) New Engl. J. Med. 333:823-831; U.S. Pat. Nos. 6,387,404 and 6,375,972).

An “effective amount” of antibody or other therapeutic, or diagnostic to be employed will depend, i.e., upon the objectives, the route of administration, the type of antibody employed, and the condition of the patient or subject. Accordingly, it will be necessary for the therapist to titer the dosage and modify the route of administration as required to obtain the optimal therapeutic effect. Typically, the clinician will administer the antibody until a dosage is reached that achieves the desired effect. The progress of this therapy is easily monitored by conventional assays. An effective amount of therapeutic will decrease the symptoms typically by at least about 10%; usually by at least about 20%; preferably at least about 30%; more preferably at least about 50%; and most preferably by at least about 90%.

As a general proposition, the initial pharmaceutically effective amount of the antibody administered parenterally will be in the range of about 0.1 μg/kg to 10 mg/kg of the patient's body weight per day, ordinarily 0.1 μg/kg/day to 1.0 mg/kg/day, preferably 0.1 μg/kg/day to 0.1 mg/kg/day, more preferably 0.1 μg/kg/day to 0.01 mg/kg/day, and most preferably 0.1 μg/kg/day, or less. The desired dosage can be delivered by a single bolus administration, by multiple bolus administrations, or by continuous infusion, depending on the pattern of pharmacokinetics that the practitioner wishes to achieve. These suggested amounts of antibody are subject to a fair amount of therapeutic discretion. The key factor in selecting an appropriate dose and scheduling is the result obtained. In the treatment and prevention of an inflammatory disorder the therapeutic composition will be formulated, dosed, and administered in a fashion consistent with good medical practice.

The “therapeutically effective amount” of antibody or binding composition to be administered will be the minimum amount necessary to prevent, ameliorate, or treat the inflammatory or proliferative disorder while minimizing possible toxic effects to the host or patient.

X. Kits.

This invention provides IL-0beta proteins, fragments thereof, nucleic acids, and fragments thereof, in a diagnostic kit. Also provided are binding compositions, including antibodies or antibody fragments, for the detection of SEQ ID NOs: 2 or 4, and metabolites and breakdown products thereof. Typically, the kit will have a compartment containing either a IL-0beta polypeptide, or an antigenic fragment thereof, a binding composition thereto, or a nucleic acid, e.g., a nucleic acid probe or primer.

The kit may comprise, e.g., a reagent and a compartment, a reagent and instructions for use, or a reagent with a compartment and instructions for use. The reagent may comprise an IL-0beta, or an antigenic fragment thereof, a binding composition, or a nucleic acid. A kit for determining the binding of a test compound, e.g., acquired from a biological sample or from a chemical library, can comprise a control compound, a labeled compound, and a method for separating free labeled compound from bound labeled compound.

Diagnostic assays can be used with biological matrices such as live cells, cell extracts, cell lysates, fixed cells, cell cultures, bodily fluids, or forensic samples. Conjugated antibodies useful for diagnostic or kit purposes, include antibodies coupled to dyes, isotopes. enzymes, and metals, see, e.g., Le Doussal, et al. (1991) New Engl. J. Med. 146:169-175; Gibellini, et al. (1998) J. Immunol. 160:3891-3898; Hsing and Bishop (1999) New Engl. J. Med. 162:2804-2811; Everts, et al. (2002) New Engl. J. Med. 168:883-889. Various assay formats exist, such as radioimmunoassays (RIA), ELISA, and lab on a chip (U.S. Pat. Nos. 6,176,962 and 6,517,234).

XI. Uses.

The invention provides agonists and antagonists of polypeptides and nucleic acids of SEQ ID NOs: 1-4, and methods for using these reagents, for the diagnosis and treatment of, e.g., inflammatory and proliferative conditions. These conditions include, e.g., rheumatoid arthritis, Alzheimer's disease, pulmonary alveolar proteinosis, melanoma, and ovarian, gastrointestinal, prostate, renal, and brain cancer, e.g., glioblastoma.

Alterations in expression or genetics of the IL-1 family of cytokines occur in inflammatory disorders of the nervous system. The inflammatory nature of Alzheimer's disease has been described (Michaelis (2003) J. Pharm. Exp. Therapeutics 304:897-904; Hoozemans, et al. (2002) Drugs Today 38:429-443; McGeer and McGeer (2001) Arch. Neurol. 58:1790-1792; Kaijzel, et al. (2002) Tissue Antigens 59:122-126; Jouvenne, et al. (1999) Eur. Cytokine Netw. 10:33-36). IL-1 family members induce expression of amyloid protein, a pathological feature of Alzheimer's diasease (Rogers, et al. (1999) J. Biol. Chem. 274:6421-6431; Donnelly, et al. (1990) Cell Mol. Neurobiol. 10:485-495; Goldgaber, et al. (1989) Proc. Natl. Acad. Sci. USA 86:7606-7610; Sheng, et al. (1994) J. Neurochem. 63:1872-1879). The invention also finds use in the diagnosis and treatment of other neurological disorders, e.g., isehemia and stroke, trauma, epilepsy, Down's syndrome, and AIDS, as IL-1 contributes to these disorders, see, e.g., Griffin and Mrak (2002) J. Leukocyte Biol. 72:233-238; Emsley and Tyrrell (2002) J. Cereb. Blood Flow Metab, 22:1399-1419; De Simoni, et al. (2002) Clin. Exp. Hypertens. 24: 535-542; Wang and Shuaib (2002) Prog. Neurobiol. 67:161-172).

IL-1 family members have been implicated in brain pathology, e.g., Alzheimer's disease, in specific regions of the brain, e.g., the frontal, occipital, temporal lobes, hippocampus, amygdala, and other regions of the brain. IL-1 family members have been found to mediate memory, Alzheimer's disease, and inflammation in these regions of the brain, see, e.g., Sheng, et al. (1995) Neuropathol. Appl. Neurobiol. 21: 290-301; Frost, et al. (2001) J. Neuroimmunol. 121:32-39; Schneider, et al. (1998) Proc. Natl. Acad. Sci. USA 95:7778-7783; Yirmiya, et al. (2002) Neurobiol. Learning Memory 78:379-389; Matsumoto, et al. (2001) European J. Pharmacol. 430:283-288; Mrak and Griffin (2001) Neurobiol. Aging 22:903-908; Cacabelos, et al. (1994) Methods Find. Exp. Clin. Pharmacol. 16:141-151; Yabuuchi, et al. (1993) Brain Res. Mol. Brain Res. 20:153-161; Eriksson, et al. (1998) Brain Res. Mol. Brain Res. 58:195-208; Ben-Ari and Cossart (2000) Trends Neurosci. 23:580-587; Wong, et al. (1997) Proc. Natl. Acad. Sci. USA 94:227-232; Rothwell and Hopkins (1995) Trends Neurosci. 18:130-136.

The invention also finds use in the treatment and diagnosis of pulmonary alveolar proteinosis, a disorder of the lungs characterized by accumulation of macrophages, fibrosis, and death (Seymour and Presneill (2002) Am. J. Respir. Crit. Care Med. 166:215-235; Carraway, et al. (2001) Am. J. Physiol. Lung Cell Mol. Physiol. 280:L377-L378).

The broad scope of this invention is best understood with reference to the following examples, which are not intended to limit the inventions to the specific embodiments.

### EXAMPLES

### I. General Methods

Standard methods of biochemistry and molecular biology are described or referenced, e.g., in Maniatis, et al. (1982) Molecular Cloning, A Laboratory Manual, Cold Spring Harbor Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.; Inis, et al. (eds.) (1990) PCR Protocols: A Guide to Methods and Applications, Academic Press, N.Y. Standard methods are also found in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4). Methods for producing fusion proteins are described. See, e.g., Invitrogen (2002) Catalogue, Carlsbad, Calif.; Amersham Pharmacia Biotech (2002), Catalogue, Piscataway, N.J.; Liu, et al. (2001) Curr. Protein Pept. Sci. 2:107-121; Graddis, et al. (2002) Curr. Pharm. Biotechnol. 3:285-297. Standard methods of histology are described (Carson (1997) Histotechnology: A Self-Instructional Text, 2^{nd} ed., Am. Soc. Clin. Pathol. Press, Chicago, Ill.; Bancroft and Gamble (eds.) (2002) Theory and Practice of Histological Techniques, 5^{th} ed., W.B. Saunders Co., Phila., Pa.).

Methods for antibody production and modification are described in, e.g., Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.); Harlow and Lane (1988) Antibodies A Laboratory Manual, Cold Spring Harbor Laboratory Press, Cold Spring Harbor, N.Y.; Einhauer, et al. (2001) J. Biochem. Biophys. Methods 49:455-465. Methods for adenovirus engineering and transfection, e.g., into cells or mammals, are described (Hurst, et al. (2002) New Engl. J. Med. 169:443-453; Danthinne and Imperiale (2000) Gene Ther. 7:1707-1714; Carlisle (2002) Curr. Op. Mol. Ther. 4:306-312.

Methods for protein purification such as immunoprecipitation, column chromatography, electrophoresis, isoelectric focusing, centrifugation, and crystallization, are described (Coligan, et al. (2000) Current Protocols in Protein Science, Vol. 1, John Wiley and Sons, Inc., New York). Chemical analysis, chemical modification, post-translational modification, and glycosylation of proteins is described. See, e.g., Coligan, et al. (2000) Current Protocols in Protein Science, Vol. 2, John Wiley and Sons, Inc., New York; Walker (ed.) (2002) Protein Protocols Handbook, Humana Press, Towota, N.J.; Lundblad (1995) Techniques in Protein Modification, CRC Press, Boca Raton, Fla. Techniques for characterizing binding interactions are described (Coligan, et al. (2001) Current Protocols in Immunology, Vol. 4, John Wiley and Sons, Inc., New York; Parker, et al. (2000) J. Biomol. Screen. 5: 77-88; Karlsson, et al. (1991) J. Immunol. Methods 145:229-240; Neri, et al. (1997) Nat. Biotechnol. 15:1271-1275; Jonsson, et al. (1991) Biotechniques 11:620-627; Friguet, et al. (1985) J. Immunol. Methods 77: 305-319; Hubble (1997) Immunol. Today 18:305-306; Shen, et al. (2001) J. Biol. Chem. 276:47311-47319).

Software packages for determining, e.g., antigenic fragments, leader sequences, protein folding, functional domains, glycosylation sites, and sequence alignments, are available, see, e.g., Vector NTI® Suite (Informax, Inc, Bethesda, Md.); GCG Wisconsin Package (Acers, Inc. San Diego, Calif.); Decipher® (Time Logic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von-Heijne (1986) Nucleic Acids Res. 14:4683-4690.

II. Cell Transfection and Expression of IL-0beta.

Human IL-0beta was subcloned in pCMV to produce the plasmid, pCMV-FLAG-hIL-0beta (Stratagene, Inc., La Jolla, Calif.). HEK293T cells (5×10⁶ cells) were transfected with 20 micrograms of pCMV encoding hIL-0beta-FLAG (pCMV-FLAG-hIL-0beta) or mIL-0beta-FLAG (pCMV-FLAG-mIL-0beta). The transfected cells were incubated for 48 h, followed by Western blot analysis of the superantants using FLAG-M2® (cat. no. 00471, Sigma-Aldrich, St. Louis, Mo.) conjugated with horse radish peroxidase (HRP). Western blot analysis demonstrated that secreted hIL-0beta and mIL-0beta migrated with an apparent molecular weight of 49 kDa. pCMV-FLAG-hIL-0beta (2.4 micrograms DNA) was transfected into HFK293T cells using 24 microliters FUGENE-6® (cat. no. 1814443, Roche Diagnostics Corp., Indianapolis Ind.) Transfected cells were incubated about 48 h, followed by collection of supernatant, purification of expressed protein with anti-FLAG-Agarose (cat. no. A-1205, Sigma-Aldrich, St. Louis, Mo.), and separation of the purified material by SDS-PAGE. Protein separated on the SDS-PAGE gel was transferred to a polvinylidene fluoride (PVDF) membrane, followed by probing of the membrane with anti-FLAG-HRP antibody (cat no. A8592, Sigma-Aldrich, St. Louis, Mo.). Development by way of the HRP assay revealed a molecular weight of about 49 kDa.

III. Expression of mRNA Encoding IL-0beta.

Human IL-0beta expression by Taqman® analysis was assessed from cDNA prepared from the indicated human cells and tissues (Table 1). (−) means <1.0, relative to ubiquitin expression. (+) means 1 to 10. (++) indicates expression within the range of 10-30. (+++) means 31-99. (++++) means 100-200. All results were normalized to ubiquitin expression. For example, expression was high in ovary carcinoma relative to normal ovary, high in rheumatoid arthritis synovia relative to control synovia, and high in colon carcinoma relative to normal colon fibroblasts (Table 1); IL-0beta expression was also high in certain regions of the brain, e.g., amygdala, cerebral cortex, frontal lobe, and hippocampus (Table 1).

TABLE 1
Relative expression of hIL-0beta mRNA.
TISSUE

| | |
|---|---|
| Control synovia. | + |
| Synovia rheumatoid arthritis | ++ |
| Normal ovary | − to + |
| Ovary carcinoma | ++ |
| Ovary papillary serous cystadenocarcinoma | ++ |
| Control lung | + |
| Lung, pulmonary alveolar proteinosis | ++ |
| Amygdala | ++++ |
| Cerebellum | − |
| Cerebral cortex | +++ |
| Corpus callosum | + |
| Frontal lobe | +++ |
| Hippocampus | +++ |
| Parietal lobe | +++ |
| Medulla | ++ |
| Occipital lobe | ++++ |
| Temporal lobe | ++++ |
| Caudate putamen | ++ |
| Substantia nigra | +++ |
| Hypothalamus | +++ |

CELL

| | |
|---|---|
| Fibroblast cell normal colon. | + |
| Colon carcinoma COLO205 cell line. | ++++ |
| Colon carcinoma HCC2998 cell line. | ++ |
| Normal epithelial cell bronchial | + to ++ |
| Normal epithelial prostate primary | ++ |
| Prostate carcinoma PC3 cell line | ++++ |
| Brain glioblastoma U251 cell line | ++++ |
| Epithelial cell renal primary cells | ++ |
| Kidney carcinoma A498 cell line | ++++ |
| Epdermal melanocyte | + to ++ |
| Skin melanoma | + to +++ |
| T cell resting | − to + |
| Monocyte | − |
| Dendritic cell | − |
| Mast cell resting | ++ |

IL-0beta expression was assessed at various times after y-irradiation (Table 2A). Maximal increases in expression occurred at, e.g., 6-12 h after irradiation (Table 2A). IL-0beta expression was also found to increase in synchronized embryonic fibroblast skin cells, e.g., at 6-12 h after release from serum starvation (Table 2B).

TABLE 2A
Expression of SEQ ID NO: 1 mRNA after γ-irradiation, relative to ubiquitin.

| Time after | 0.5 h | 1.0 h | 1.5 h | 2.0 h | 4.0 h | 6.0 h | 8.0 h | 10 h | 12 h | 24 h | 48 h |
|---|---|---|---|---|---|---|---|---|---|---|---|
| irradiation | | | | | | | | | | | |
| (hours). | | | | | | | | | | | |
| Expression | 0.2 | 1.5 | 1.3 | 1.1 | 8.9 | 24.1 | 13.5 | 9.1 | 20.5 | 4.6 | 6.1 |

TABLE 2B
Expression of SEQ ID NO: 1 mRNA after release from serum starvation,
relative to ubiquitin. “Asyn. cells” means asynchronous cells.

| | | | | | | | | | | |
|---|---|---|---|---|---|---|---|---|---|---|
| Time after | 0 h | 3 h | 6 h | 9 h | 12 h | 15 h | 18 h | 24 h | 36 h | Asyn. |
| release (h) | | | | | | | | | | cells |
| Expression | 3.4 | 4.0 | 10.3 | 5.1 | 5.1 | 3.3 | 2.9 | 2.8 | 2.3 | 2.9 |
| Percent G1 phase (%) | 91.5 | 94 | 93.6 | 94 | 95 | 94.6 | 94.6 | 89.1 | 72 | 75 |
| Percent S phase (%) | 2 | 1.6 | 1.7 | 0.6 | 0.8 | 0.6 | 0.7 | 5.2 | 12.6 | 11.5 |
| Percent | 3.5 | 3.6 | 4.4 | 4.9 | 4.9 | 4.5 | 4.6 | 5.7 | 15.3 | 13 |
| G2/M | | | | | | | | | | |
| phase (%) | | | | | | | | | | |

Many modifications and variations of this invention, as will be apparent to one of ordinary skill in the art can be made to adapt to a particular situation, material, composition of matter, process, process step or steps, to preserve the objective, spirit, and scope of the invention. All such modifications are intended to be within the scope of the claims appended hereto without departing from the spirit and scope of the invention. The specific embodiments described herein are offered by way of example only, and the invention is to be limited by the terms of the appended claims, along with the fill scope of the equivalents to which such claims are entitled; and the invention is not to be limited by the specific embodiments that have been presented herein by way of example.

### DESCRIPTION OF SEQUENCE LISTING

SEQ ID NO: 1 is human IL-0beta (hIL-0beta) nucleic acid.

SEQ ID NO: 2 is human IL-0beta polypeptide.

SEQ ID NO: 3 is mouse IL-0beta (mIL-0beta) nucleic acid.

SEQ ID NO: 4 is mouse mIL-0beta polypeptide.

SEQ ID NO: 5 is human IL-1 alpha.

SEQ ID NO: 6 is human IL-1beta.

SEQ ID NO: 7 is human basic-fibroblast growth factor (basic-FGF).

What is claimed is:

1. An isolated polypeptide comprising SEQ ID NOs: 2 or 4, or an antigenic fragment thereof.

2. The polypeptide of claim 1, further comprising a fusion polypeptide or fusion peptide.

3. A isolated nucleic acid encoding the polypeptide of claim 1.

4. An expression or replicating vector comprising the nucleic acid of claim 3.

5. A host cell comprising the vector of claim 4.

6. A binding composition that specifically binds to the polypeptide of claim 1.

7. The binding composition of claim 6, derived from the antigen binding site of an antibody.

8. The binding composition of claim 7, comprising:
a) a human antibody;
b) a humanized antibody;
c) a monoclonal antibody;
d) a polyclonal antibody;
e) an Fab fragment or F(ab′)₂ fragment; or
f) a detectable label.

9. A method of producing a polypeptide comprising SEQ ID NOs: 2 or 4, or an antigenic fragment thereof, comprising:
a) culturing the host cell of claim 5 under conditions suitable for expression of the polypeptide; and
b) isolating or purifying the polypeptide.

10. A method of modulating the activity of a cell comprising contacting the cell with:
a) an agonist of SEQ ID NOs: 2 or 4; or
b) an antagonist of SEQ ID NOs: 2 or 4.

11. The method of claim 10, wherein the modulating is inhibiting.

12. The method of claim 10, wherein the agonist or antagonist comprises a binding composition derived from the antigen-binding site of an antibody that specifically binds to SEQ ID NOs: 2 or 4, or an antigenic fragment thereof.

13. A method of treating a subject suffering from a cellular disorder comprising treating with or administering an effective amount of:
a) an agonist of SEQ ID NOs: 2 or 4; or
b) an antagonist of SEQ ID NOs: 2 or 4.

14. The method of claim 13, wherein the agonist or antagonist comprises a binding composition derived from the antigen-blinding site of an antibody that specifically binds SEQ ID NOs: 2 or 4, or an antigenic fragment thereof.

15. The method of claim 14, wherein the binding composition comprises:
a) a human antibody;
b) a humanized antibody;
c) a monoclonal antibody;
d) a polyclonal antibody;
e) an Fab fragment or F(ab′)₂ fragment; or
f) a detectable label.

16. The method of claim 13, wherein the cellular disorder is,
a) an immune or inflammatory disorder; or
b) a proliferative condition.

17. The method of claim 13, wherein the cellular disorder is:
a) rheumatoid arthritis; or
b) cancer.

18. The method of claim 17, wherein the cancer is ovarian cancer, melanoma, renal cancer, prostate cancer, or gastrointestinal cancer.

19. A method of diagnosing a cellular disorder comprising contacting a sample from a subject with:
a) the binding composition of claim 6; or
b) a nucleic acid that specifically hybridizes to a polynucleotide comprising SEQ ID NOs: 1 or 3.

20. A kit comprising:
a) the binding composition of claim 6, or a nucleic acid that specifically hybridizes to a nucleic acid comprising SEQ ID NOs: 1 or 3; and
b) instructions for use.
